(19) United States
(12) Patent Application Publication
(10) Pub. No.: US 2025/0099725 A1
(43) Pub. Date: Mar. 27, 2025

(54) Fixturing to Facilitate Drug Coating of Long Angioplasty Balloons

(71) Applicant: Boston Scientific Scimed, Inc., Maple Grove, MN (US)

(72) Inventor: Louis Warner Stefanich, Minneapolis, MN (US)

(73) Assignee: Boston Scientific Scimed, Inc., Maple Grove, MN (US)

(21) Appl. No.: 18/897,077

(22) Filed: Sep. 26, 2024

Related U.S. Application Data
(60) Provisional application No. 63/540,918, filed on Sep. 27, 2023.

(51) Int. Cl. A61M 25/10

(52) U.S. Cl. CPC A61M 25/104; A61M 2025/1075

(57) ABSTRACT

A fixture for holding a medical device during a coating process. The fixture may comprise a mounting rail extending from a first end region to a second end region, a drive unit movably coupled to the mounting rail, a proximal coupling assembly removably coupled to the drive unit, a support member movably coupled to the mounting rail, a distal coupling assembly removably coupled to the support member, and a mandrel configured to extend through a lumen of a medical device. The mandrel may have a first end configured to be releasably coupled to the proximal coupling assembly and a second end configured to be releasably coupled to the distal coupling assembly. Actuation of the distal coupling assembly and/or the support member may be configured to pull the mandrel taut.

### CROSS-REFERENCE TO RELATED APPLICATIONS

This application claims the benefit of priority under 35 U.S.C. § 119 of U.S. Provisional Application No. 63/540,918, filed Sep. 27, 2023, the entire disclosure of which is hereby incorporated by reference.

### TECHNICAL FIELD

The present disclosure pertains to the manufacturing of medical devices and more particularly to the manufacturing of medical devices that include a therapeutic coating.

### BACKGROUND

Balloon catheters may be used to widen or enlarge passages in the body, such as, but not limited to, within the vasculature. In some instances, it may be desirable to treat long vessel sections with a single balloon. It may be further desirable to coat, layer, or otherwise apply an elutable drug or therapeutic agent to an outer surface of the balloon to deliver and/or administer the drug or therapeutic agent to a lumen wall when the balloon is expanded. However, balloons of a long length may sag in the middle as a drug coating is applied thereto. Of the known medical devices, systems, and methods, each has certain advantages and disadvantages. There is an ongoing need to provide alternative medical devices and systems, including devices and systems for coating long balloons.

### SUMMARY

The present disclosure pertains to medical devices and more particularly to systems and methods for coating a balloon of a catheter.

In a first example, a fixture for holding a medical device may comprise a mounting rail extending from a first end region to a second end region, a drive unit movably coupled to the mounting rail adjacent the first end region thereof, a proximal coupling assembly removably coupled to the drive unit, a support member movably coupled to the mounting rail adjacent the second end region thereof, a distal coupling assembly removably coupled to the support member, and a mandrel configured to extend through a lumen of a medical device. The mandrel may have a first end configured to be releasably coupled to the proximal coupling assembly and a second end configured to be releasably coupled to the distal coupling assembly. The proximal coupling assembly may comprise a first connection member, a second connection member releasably coupled to the first connection member, and a collet disposed within a lumen of the first connection member and within a lumen of the second connection member. The distal coupling assembly may comprise a chuck, a cap, a body, and an elongate member.

Alternatively or additionally to any of the examples above, in another example, the mandrel may extend through the lumen of the second connection member and a lumen of the collet.

Alternatively or additionally to any of the examples above, in another example, when the first connection member is coupled with the second connection member, the second connection member may exert a radially compressive force on the collet.

Alternatively or additionally to any of the examples above, in another example, when the first connection member is coupled with the second connection member, the collet may exert a radially compressive force on an outer surface of the mandrel.

Alternatively or additionally to any of the examples above, in another example, the collet may have a first outer diameter adjacent to a first end thereof and a second outer diameter adjacent a second end thereof, the second outer diameter greater than the first outer diameter.

Alternatively or additionally to any of the examples above, in another example, a distal end region of the collet may comprise a plurality of radially actuated segments.

Alternatively or additionally to any of the examples above, in another example, the second end of the mandrel may be configured to be received within the chuck of the distal coupling assembly.

Alternatively or additionally to any of the examples above, in another example, the support member may comprise a mounting assembly movably coupled to an actuatable knob.

Alternatively or additionally to any of the examples above, in another example, actuation of the actuatable knob may be translated to linear moment of the mounting assembly.

Alternatively or additionally to any of the examples above, in another example, actuation of the actuatable knob in a first direction may be configured to move the distal coupling assembly towards the second end region of the mounting rail and actuation of the actuatable knob in a second direction opposite the first direction may be configured to move the distal coupling assembly towards the first end region of the mounting rail.

Alternatively or additionally to any of the examples above, in another example, the support member may be axially displaced along the mounting rail to pull the mandrel taut.

Alternatively or additionally to any of the examples above, in another example, the mounting assembly may be axially displaced along an axis parallel to the mounting rail to pull the mandrel taut.

Alternatively or additionally to any of the examples above, in another example, the drive unit may be configured to actively rotate the proximal coupling assembly.

Alternatively or additionally to any of the examples above, in another example, the first connection member may comprise a female luer lock adjacent a first end thereof and a male luer lock adjacent a second end thereof.

Alternatively or additionally to any of the examples above, in another example, the second connection member may comprise a female luer lock adjacent a first end thereof and a male luer lock adjacent a second end thereof.

In another example, a fixture for holding a medical device may comprise a mounting rail extending from a first end region to a second end region, a drive unit movably coupled to the mounting rail adjacent the first end region thereof, a proximal coupling assembly removably coupled to the drive unit, a support member movably coupled to the mounting rail adjacent the second end region thereof, a distal coupling assembly removably coupled to the support member, and a mandrel configured to extend through a lumen of a medical device. The mandrel may have a first end configured to be releasably coupled to the proximal coupling assembly and a second end configured to be releasably coupled to the distal coupling assembly. The proximal coupling assembly may comprise a first connection member, a second connection member releasably coupled to the first connection member, and a collet disposed within a lumen of the first connection member and within a lumen of the second connection member. Actuation of the distal coupling assembly and/or the support member may be configured to pull the mandrel taut.

Alternatively or additionally to any of the examples above, in another example, when the first connection member is coupled with the second connection member, the collet may exert a radially compressive force on an outer surface of the mandrel.

In another example, a fixture for holding a medical device may comprise a mounting rail extending from a first end region to a second end region, a drive unit movably coupled to the mounting rail adjacent the first end region thereof, a proximal coupling assembly removably coupled to the drive unit, a support member movably coupled to the mounting rail adjacent the second end region thereof, a distal coupling assembly removably coupled to the support member, and a mandrel configured to extend through a lumen of a medical device. The proximal coupling assembly may comprise a first connection member, a second connection member releasably coupled to the first connection member, and a collet disposed within a lumen of the first connection member and within a lumen of the second connection member. The distal coupling assembly may comprise a chuck, a cap, a body, and an elongate member. The mandrel may have a first end configured to be releasably coupled to the proximal coupling assembly and a second end configured to be releasably coupled to the distal coupling assembly. Actuation of the distal coupling assembly and/or the support member may be configured to pull the mandrel taut.

Alternatively or additionally to any of the examples above, in another example, the first connection member may comprise a female luer lock adjacent a first end thereof and a male luer lock adjacent a second end thereof.

Alternatively or additionally to any of the examples above, in another example, the lumen of the first connection member may have a varying diameter along a length thereof.

Alternatively or additionally to any of the examples above, in another example, the second connection member may comprise a female luer lock adjacent a first end thereof and a male luer lock adjacent a second end thereof.

Alternatively or additionally to any of the examples above, in another example, the lumen of the second connection member may have a first diameter adjacent a proximal end thereof and a second diameter adjacent to a distal end thereof, the first diameter greater than the second diameter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example drug delivery balloon catheter;

FIG. 2 is a cross-sectional view of a catheter-based balloon taken through line 2-2 in

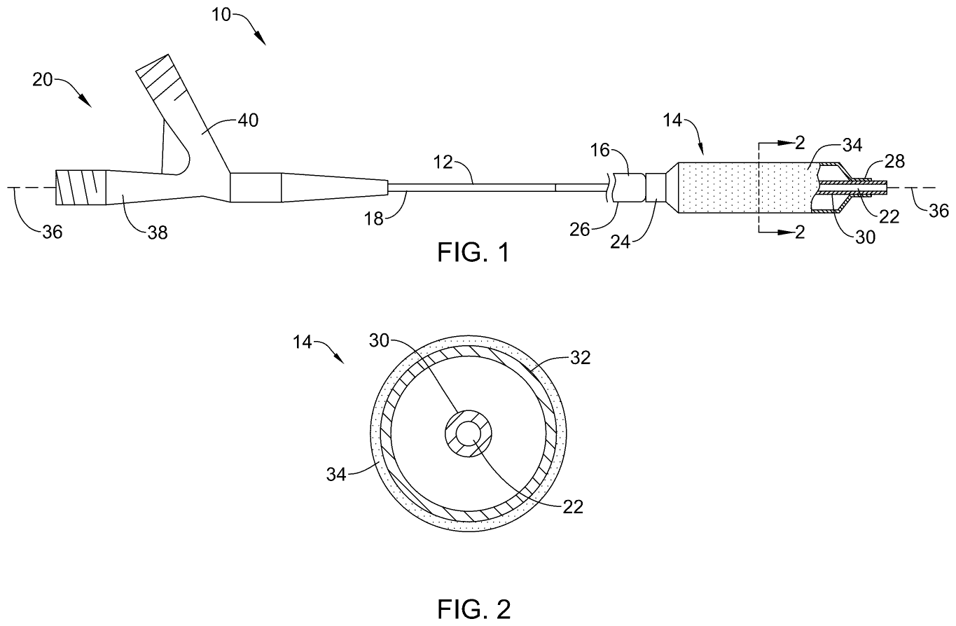

FIG. 1;

FIG. 3 is a schematic view of an illustrative system for coating a balloon;

FIG. 4 is an unassembled view of the proximal end region of the catheter and the coupling assembly;

FIGS. 5-7 illustrate side views of the proximal end region of the catheter and the coupling assembly as the coupling assembly is assembled and connected to the catheter;

FIG. 8 is a side view of the coupling assembly coupled to the drive unit;

FIG. 9 is a perspective view of the distal end region of the catheter secured to the second end region of the fixture; and

FIG. 10 illustrates a partially exploded perspective view of the illustrative bearings and chuck assembly.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The terms “therapeutic agents,” “drugs,” “bioactive agents,” “pharmaceuticals,” “pharmaceutically active agents”, and other related terms may be used interchangeably herein and include genetic therapeutic agents, non-genetic therapeutic agents, and cells. Therapeutic agents may be used singly or in combination. A wide range of therapeutic agent loadings can be used in conjunction with the devices of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

The following detailed description should be read with reference to the drawings in which similar elements in different drawings are numbered the same. The drawings, which are not necessarily to scale, depict illustrative embodiments and are not intended to limit the scope of the invention.

Balloon catheters may be used to widen or enlarge passages in the body, such as, but not limited to, within the vasculature. In some instances, it may be desirable to treat long vessel sections with a single, long balloon. It is further contemplated that it may be desirable to provide a therapeutic coating on the balloon that may reduce thrombus development and/or growth. However, long balloons (e.g., a balloon having a length of about 200 millimeters or more) may be difficult to coat with the therapeutic coating as the product mandrel may not provide enough support to prevent sagging in the middle of the balloon. In some cases, shorter balloons (e.g., having a length of less than about 200 millimeters) may also sag in the middle of the balloon. In one example, larger diameter balloons may have an increased mass which may contribute to sagging in the middle of the balloon. Sagging of the balloon may in turn cause excessive balloon wobble or uneven rotation as the balloon catheter is rotated during coating. During the coating process a tube or nozzle may be passed over the surface of the folded balloon and the coating solutions are deposited through the nozzle. One aspect of this coating process, which helps to achieve a uniform coating, is the ability to maintain a “liquid bridge” or a continuous connection of the coating material between the nozzle and the balloon surface. The sagging and resulting wobble of various balloons may prevent or inhibit the maintenance of the liquid bridge as the coating machine, and thus the nozzle, may not have the ability to follow the balloon surface as the coating machine travels back and forth. For example, the coating machine may be limited to movement along a single axis. The present disclosure is directed towards systems and methods for coating balloons which prevent the balloon from sagging during the coating process. While the systems and methods are described with respect to long balloons, the systems and methods described herein may be used to coat balloons of any length, or other devices, such as, but not limited to, stents, endoluminal implants, transluminal implants, other drug transfer devices, and the like. Further, the systems and methods described herein may be used to apply coatings other than therapeutic agents to various medical devices.

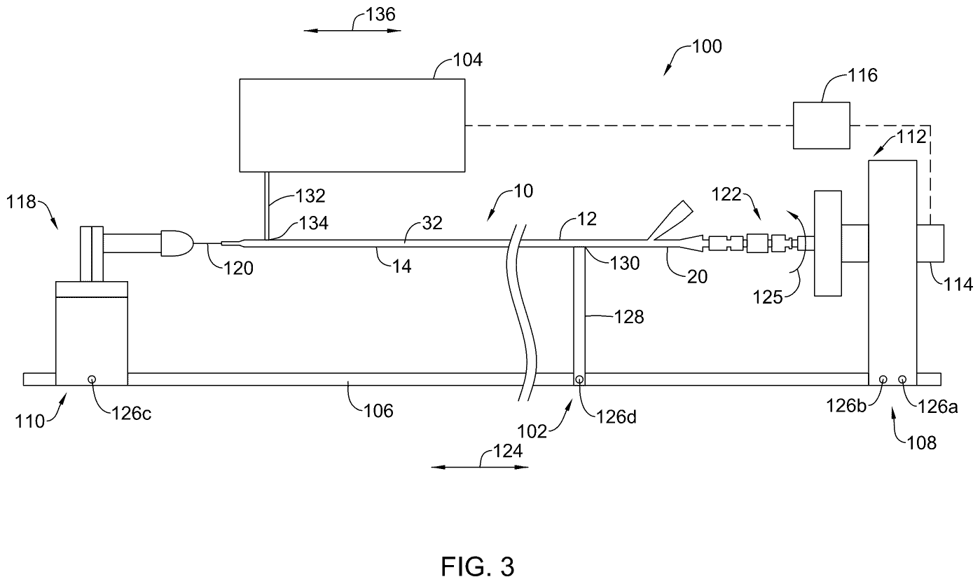

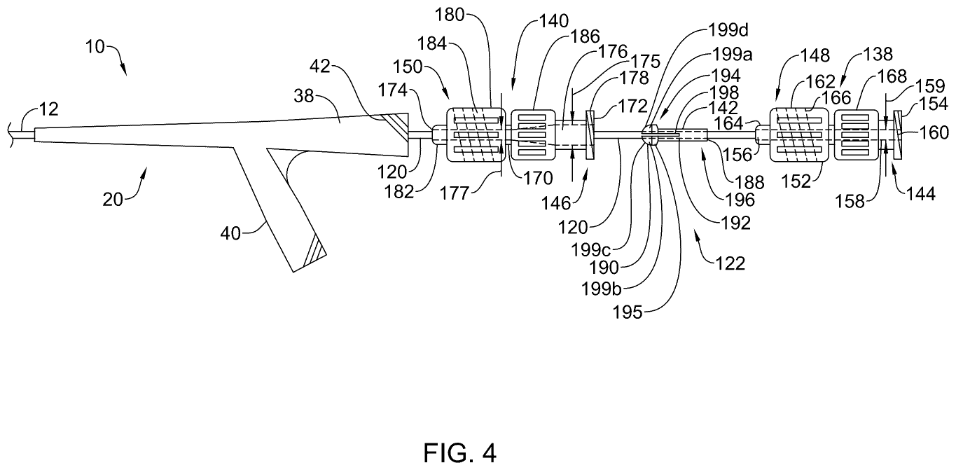

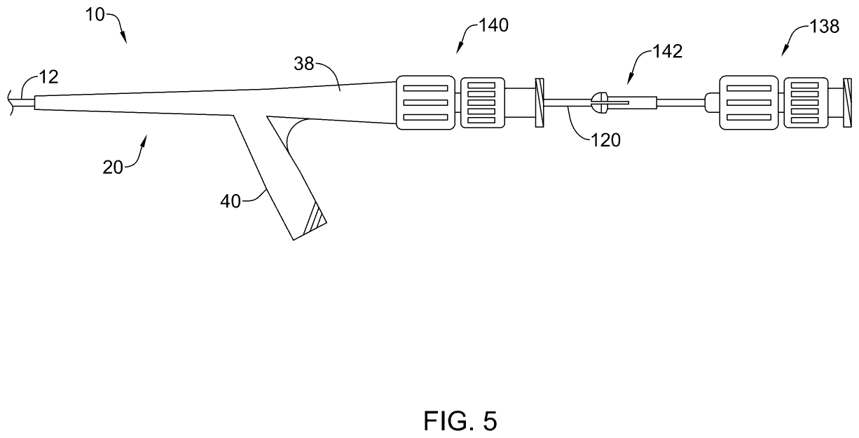

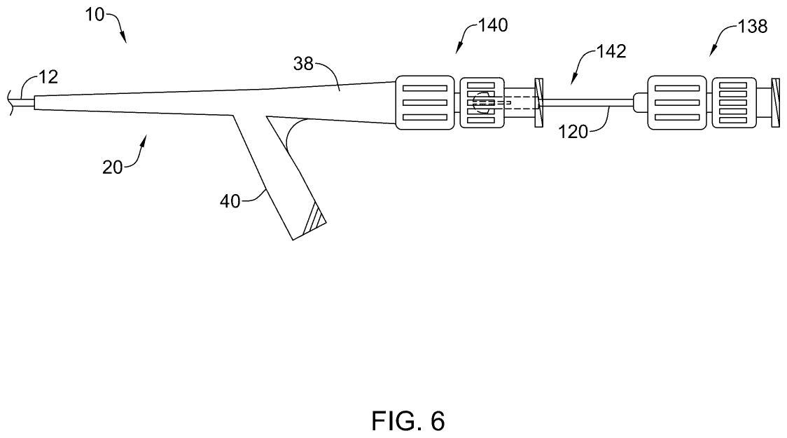

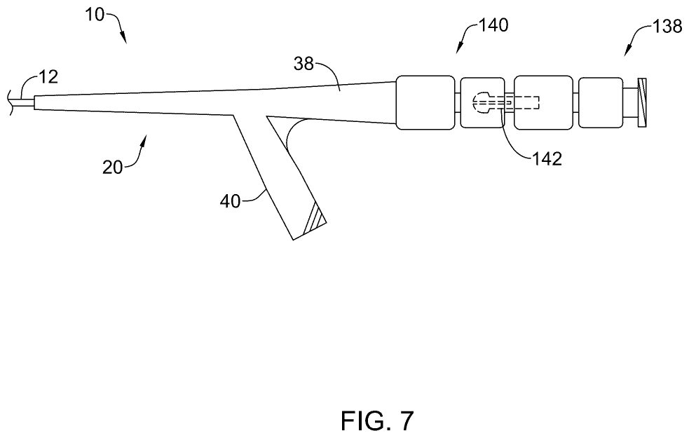

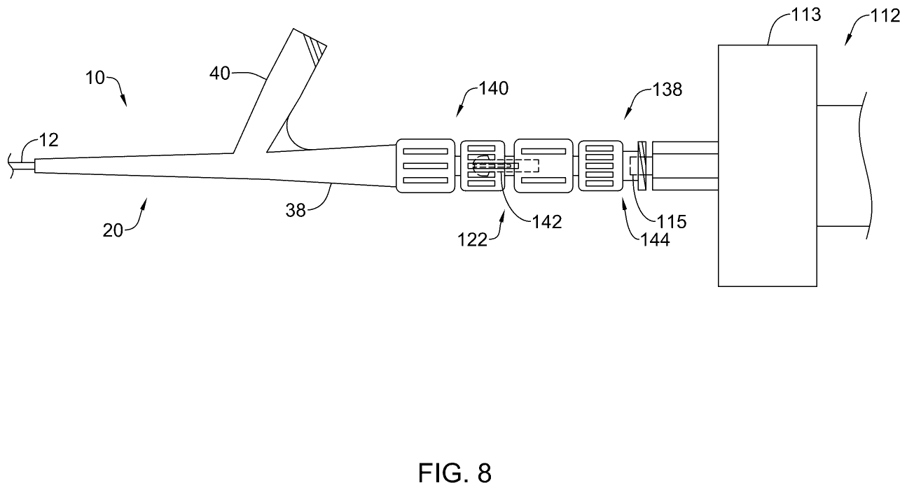

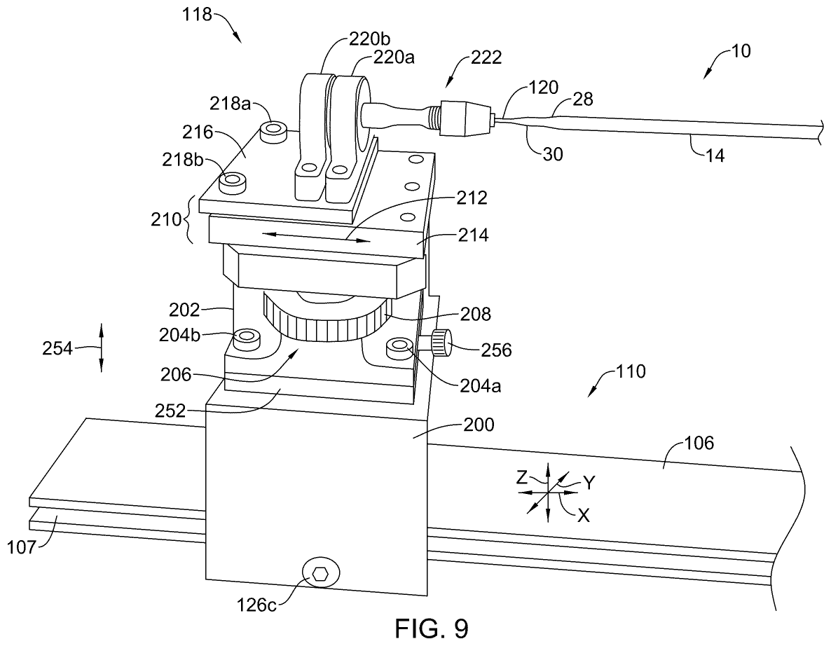

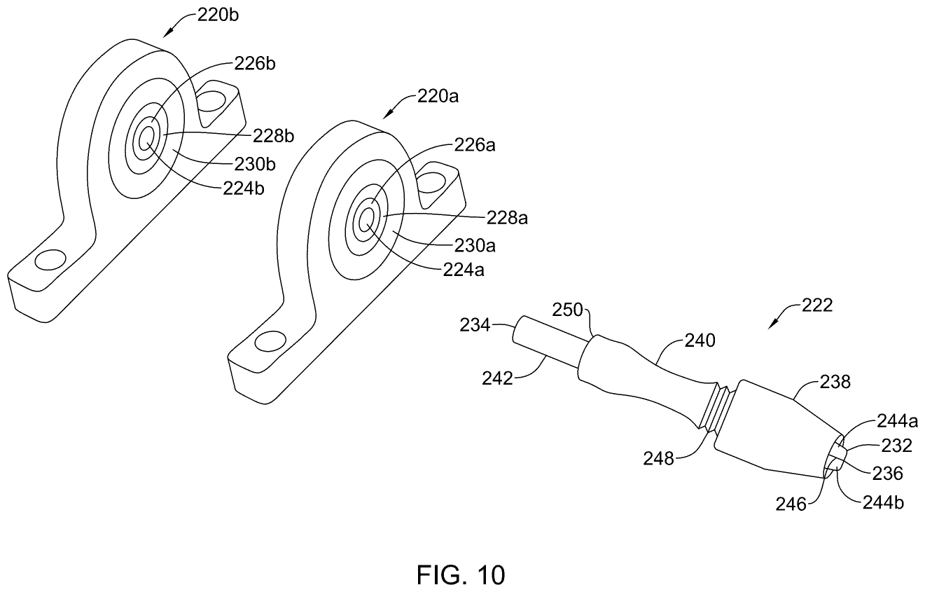

FIG. 1 is a schematic side view of a drug delivery balloon catheter 10. A cross-sectional view of the drug delivery balloon catheter 10 is shown in FIG. 2. In the illustrated embodiment, the catheter 10 may include an elongated shaft 12, an inflatable balloon 14 coupled at or to a distal portion 16 of the shaft 12, along with other components. The elongated shaft 12 may extend along a longitudinal axis 36 of the catheter 10. The elongated shaft 12 may include a tubular member having a proximal portion 18, and one or more lumens extending between the proximal portion 18 and the distal portion 16. The elongated shaft 12 may be configured to have a substantially circular cross-section; however, it may be configured to have other suitable cross-sectional shapes, such as elliptical, oval, polygonal, irregular, etc. In addition, the elongated shaft 12 may be flexible along its entire length or adapted for flexure only along portions of its length. The required degree of flexibility of the elongated shaft 12 may be predetermined based on its intended navigation to a target vascular passage, and the amount of inertial force required for advancing the elongated shaft 12 through the vascular passage. The catheter 10 may be configured as an over-the-wire (OTW) catheter, a single-operator exchange (SOE) catheter, a fixed wire catheter, and/or the like.

The cross-sectional dimensions of the elongated shaft 12 may vary according to the desired application. Generally, the cross-sectional dimensions of the elongated shaft 12 may be sized smaller than the typical blood vessel in which the catheter 10 is to be used. The length of the elongated shaft 12 may vary according to the location of the vascular passage where drug delivery is desired. In some instances, a 6F or a 5F catheter may be used as the elongated shaft 12, where “F,” also known as French catheter scale, is a unit to measure catheter diameter (1F=⅓ millimeter (mm)). In addition, the elongated shaft 12 or a portion thereof may be selectively steerable. Mechanisms such as, pull wires and/or other actuators may be used to selectively steer the elongated shaft 12, if desired.

The proximal portion 18 of the elongated shaft 12 may include a handle 20 usable to manually maneuver the distal portion 16 of the elongated shaft 12. The handle 20 may include one or more ports that may be used to introduce any suitable medical device, fluid or other interventions. For example, the handle 20 may include a guidewire port 38 in communication with a guidewire lumen 22 (shown in the cut-away portion at the distal end of the catheter 10 and also in FIG. 2) which may be used to introduce a guidewire having an appropriate thickness into the elongated shaft 12, which may guide the elongated shaft 12 to the target location within an artery. Furthermore, the handle 20 may include an inflation port 40 configured to be coupled to a source of inflation fluid for delivering an inflation fluid through an inflation lumen of the catheter shaft 12 to the inflatable balloon 14. In certain embodiments, the elongated shaft 12 may include one or more additional lumens, which may be configured for a variety of purposes, such as delivering medical devices or for providing fluids, such as saline, to a target location.

The inflatable balloon 14 may have a length that is in the range of about 200 millimeters (mm) or greater. However, this is not required. In some cases, the inflatable balloon 14 may have a length of less than 200 mm. The length of the inflatable balloon 14 may depend on the target treatment location. The inflatable balloon 14 may be operably coupled at or to the distal portion 16 of the elongated shaft 12. In particular, a proximal portion or waist 24 of the inflatable balloon 14 may be secured to the distal portion 16 of the elongated shaft 12, such as an outer tubular member 26 of the elongated shaft 12. Furthermore, a distal portion or waist 28 of the inflatable balloon 14 may be secured to the distal portion 16 of the elongated shaft 12, such as an inner tubular member 30 of the elongate shaft 12 extending through the outer tubular member 26. A suitable securing method(s) may be employed to couple the two structures, including but not limited to adhesive bonding, thermal bonding (e.g., hot jaws, laser welding, etc.) or other bonding technique, as desired. The inflatable balloon 14 may be configured to be expanded from a deflated state to an inflated state through delivery of an inflation fluid (e.g., saline) through the inflation lumen of the catheter shaft 12. The balloon 14 may be deflated during introduction of the catheter inside the patient's body and the balloon 14 may be inflated once it reaches the target site within the body vessel.

The inflatable balloon 14 may be manufactured using or otherwise formed of any suitable material, including polymer materials, such as polyamide, polyether block amide (PEBA), polyester, nylon, etc. The inflatable balloon 14 may have a substantially cylindrical configuration with a circular cross-section, as shown in the illustrative embodiment. However, in other embodiments the inflatable balloon 14 may have another suitable configuration or shape, if desired.

The inflatable balloon 14 may include a balloon wall 32 having a drug coating 34 disposed thereon. The drug coating 34 may be disposed along substantially the entire length and/or circumference of the balloon 14 or along one or more portions of the balloon 14. For example, the drug coating 34 may be disposed along a central or body portion of the balloon 14. The drug coating 34 disposed on the balloon 14/balloon wall 32 may have an average thickness in the range of about 1 micrometer (μm) to about 50 μm, for example.

The drug coating 34 may include one or more therapeutic agents such as, but not limited to, anti-thrombotic agents, anti-proliferative agents, anti-inflammatory agents, direct oral anticoagulants (DOACs), anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms. More specific drugs or therapeutic agents include paclitaxel, rapamycin, sirolimus, everolimus, tacrolimus, heparin, diclofenac, aspirin, Epo D, dexamethasone, estradiol, halofuginone, cilostazol, geldanamycin, apixaban, rivaroxaban, edoxaban, dabigatran, betrixaban, argatroban, ABT-578 (Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others, and/or combinations thereof.

FIG. 3 is a schematic view of an illustrative system 100 for applying a therapeutic agent to an outer surface of an inflatable balloon 14. Generally, the system 100 may include a fixture 102 for holding and/or rotating the catheter 10 and a coating system 104. The fixture 102 may include a mounting rail 106 extending from a first or proximal end region 108 to a second or distal end region 110. The mounting rail 106 may include channels or grooves 107 formed in the lateral sides thereof (see, for example, FIG. 9). The channels or grooves 107 may allow components coupled to the mounting rail 106 to move axially along the mounting rail 106, as shown at arrow 124 while preventing side-to-side (lateral) movement or up and down movement of the components. Said differently, the channels or grooves 107 may allow components coupled to the mounting rail 106 to move axially along an X axis while preventing or reducing movement along the Y or Z axis.

The fixture 102 may further include a drive unit 112 movably coupled to the mounting rail 106 adjacent the first end region 108 thereof. For example, the drive unit 112 may be axially displaced along the mounting rail 106 in a direction generally parallel to a longitudinal axis of the mounting rail 106, as shown at arrow 124. The drive unit 112 may be releasably secured at a desired axial location using one or more fixation mechanisms 126a, 126b. Illustrative fixation mechanisms 126a, 126b may include, but are not limited to, set screws, screws, bolts, mating pins and apertures, and the like. While not explicitly shown, the drive unit 112 may include inwardly extending protrusions adjacent the one or more fixation mechanisms 126a, 126b. The protrusions may be configured to mate with and slide within the channels 107 of the mounting rail 106.

Generally, the drive unit 112 may be configured to rotate the catheter 10 about the longitudinal axis 36 of the catheter 10, as shown at arrow 125. The drive unit 112 may include a motor 114 which is operably connected (e.g., via a wired or wireless connection) to a control unit 116. The control unit 116 may provide power to the drive unit 112 and/or control a rotational speed of the motor 114, among other variables. As will be described in more detail herein, the drive unit 112 may be releasably coupled to the proximal end region of the catheter 10 via a proximal coupling assembly 122. In some cases, the drive unit 112 may be releasably coupled to the handle 20 of the catheter 10.

The fixture 102 may further include a distal support member 118 movably coupled to the mounting rail 106 adjacent to the second end region 110 thereof. For example, the distal support member 118 may be axially displaced along the mounting rail 106 in a direction generally parallel to a longitudinal axis of the mounting rail 106, as shown at arrow 124. The distal support member 118 may be releasably secured at a desired axial location using one or more fixation mechanisms 126c. Illustrative fixation mechanisms 126c may include, but are not limited to, set screws, screws, bolts, mating pins and apertures, and the like. While not explicitly shown, the distal support member 118 may include inwardly extending protrusions adjacent the one or more fixation mechanisms 126c. The protrusions may be configured to mate with and slide within the channels 107 of the mounting rail 106.

The distal support member 118 may be releasably coupled to a distal end of a mandrel 120. The mandrel 120 may be sized and shaped to extend from a point distal to the distal end of the catheter 10 proximally through the catheter 10 (in some cases through the guidewire lumen 22) and to the proximal coupling assembly 122. A proximal end of the mandrel 120 may be releasably coupled to the proximal coupling assembly 122.

The fixture 102 may further include one or more intermediate support members 128 movably coupled to the mounting rail 106 at one or more axial locations between the handle 20 of the catheter 10 and the proximal waist 24 of the balloon 14. For example, the intermediate support members 128 may be axially displaced along the mounting rail 106 in a direction generally parallel to a longitudinal axis of the mounting rail 106, as shown at arrow 124. The intermediate support members 128 may be releasably secured at a desired axial location using one or more fixation mechanisms 126d. Illustrative fixation mechanisms 126d may include, but are not limited to, set screws, screws, bolts, mating pins and apertures, and the like. While not explicitly shown, the one or more intermediate support members 128 may include inwardly extending protrusions adjacent the one or more fixation mechanisms 126d. The protrusions may be configured to mate with and slide within the channels 107 of the mounting rail 106.

The intermediate support members 128 may be configured to support the elongated shaft 12 of the catheter 10 while allowing rotation thereof. In some cases, the intermediate support members 128 may include a v-block (not explicitly shown) adjacent a top end 130 thereof. The elongated shaft 12 may rest in the vertex of the v-block to allow rotation of the elongated shaft 12 while maintaining the longitudinal axis 36 of the catheter 10 at a desired height and lateral orientation. While FIG. 3 illustrates one intermediate support member 128, the fixture 102 may include more than one intermediate support member 128, such as, two, three, four, five, six, seven, eight, or more intermediate support members 128. It is contemplated that the number of intermediate support members 128 used may be selected, at least in part, based on a length of the elongated shaft 12. For example, longer elongated shafts 12 may require more intermediate support members 128 than shorter elongated shafts 12. The balloon 14 may be free from intermediate support members 128 to prevent the applied coating from being disturbed or disrupted by contact with an intermediate support member 128.

The drive unit 112, the distal support member 118, and the one or more intermediate support members 128 may be axially displaced along the mounting rail 106 to allow the system 100 to accommodate catheters 10 of differing lengths. Any of the drive unit 112, the distal support member 118 and/or the one or more intermediate support members 128 may be moved axially in a first direction towards the first end region 108 or in a second direction towards the second end region 110. The mounting rail 106 may be long enough such that a catheter 10 having a length of up to about 150 centimeters, or greater, may be supported within the fixture 102. The mounting rail 106 may have a length greater than the length of the catheter 10 to allow the drive unit 112 and the distal support member 118 to be coupled to the mounting rail 106 while the drive unit 112 and the distal support member 118 are also coupled to the catheter 10 proximal to the proximal end of the catheter 10 and distal to the distal end of the catheter 10, respectively.

The coating system 104 may be configured to dispense the coating solution through a nozzle or tube 132. A free end 134 of the nozzle 132 may be positioned in close proximity to the balloon 14 so that a liquid bridge of the coating solution may extend continuously (e.g., without or free from breaks, gaps, or interruptions) between the free end 134 of the dispensing nozzle 132 and the exterior surface 32 of the balloon 14. The coating system 104 may be configured to move axially in a direction parallel to the longitudinal axis 36 of the catheter 10, as shown at arrow 136. The coating system 104 may have a travel length that is at least as long as a length of the balloon 14 of the catheter 10 to allow the coating solution to be applied over an entirety of the length of the balloon 14. However, the coating solution may be applied over less than an entirety of the length of the balloon 14, as desired. The coating system 104 may be operably connected (e.g., via a wired or wireless connection) to the control unit 116. In some cases, separate control units may be provided to control the drive unit 112 and the coating system 104 independently. The control unit 116 may control a direction of travel and/or a speed of travel of the coating system 104. Further, the control unit 116 may control a dispensing volume and/or dispensing rate of the coating solution. The catheter 10 may rotate as the coating solution is dispensed to coat an entirety of the circumference of the balloon 14. However, this is not required.

FIG. 4 is an unassembled view of the proximal end region of the catheter 10 and the proximal coupling assembly 122. The proximal coupling assembly 122 may be a part of the fixture 102 and may couple the proximal end region of the catheter 10 to the drive unit 112. The proximal coupling assembly 122 may include a first or proximal connection member 138, a second or distal connection member 140, and a collet 142 disposed between the first and second connection members 138, 140. In some embodiments, the first and/or second connection members 138, 140 may be a luer coupling mechanism. However, this is not required. The first and second connection members 138, 140 may take other structures, as desired. In some examples, the first and second connection members 138, 140 may each include a female luer lock 144, 146 and a male luer lock 148, 150. The first connection member 138 and/or the second connection member 140 may be formed of any material, such as, but not limited to, nylons, polycarbonates, polyether block amides (e.g., such as, but not limited to, PEBAX®), urethanes, etc.

The first connection member 138 may include a body portion 152 extending from a first or proximal end 154 to a second or distal end 156. The body portion 152 may be formed as a single monolithic structure or may be formed from two or more components coupled to one another. A lumen 158 may extend through the first connection member 138 from the proximal end 154 to the distal end 156 thereof. The lumen 158 may have a substantially uniform diameter along the length thereof. In some cases, the diameter 159 of the lumen 158 may be about 0.125 inches (3.175 mm). It is contemplated that the diameter 159 of the lumen 158 is not limited to a particular size and may be sized to receive a portion of the collet 142 and/or a portion of the drive unit 112. In some cases, the diameter 159 of the lumen 158 may be larger than a lumen of a standard luer fitting to allow a portion of the collet 142 to be disposed within the lumen 158. In yet another embodiment, the lumen 158 may have a diameter that varies along the length thereof. For example, a proximal end region of the lumen 158 may be greater than about 0.125 inches (3.175 mm). In some examples, a diameter of the lumen 158 along the proximal end region may reduce from a first diameter adjacent to the proximal end 154 to a second diameter at an intermediate location between the proximal end 154 and the distal end 156 in a sloped or tapered manner. The first diameter may be greater than 0.125 inches (3.175 mm) while the second diameter may be approximately 0.125 inches (3.175 mm) or less than 0.125 inches (3.175 mm). The diameter of the lumen 158 extending distally from the intermediate location may be approximately 0.125 inches (3.175 mm), or sized to receive a portion of the collet 142 therein.

The first connection member 138 may include one or more threads 160 extending radially from an outer surface of the body portion 152. In some embodiments, the one or more threads 160 may be adjacent to the proximal end 154 of the body portion 152. In some examples, the one or more threads 160 may be interrupted or discontinuous. In other examples, the one or more threads 160 may be helically continuous about the body portion 152. While the first connection member 138 is illustrated as having a single thread 160, in some examples, the first connection member 138 may have less than one thread (e.g., zero, less than one, etc.) or more than one thread 160, as desired. In some cases, the proximal end region of the first connection member 138 may form a female luer connection 144.

The first connection member 138 may further include an outer coupling member 162 and an inner tubular member 164 adjacent to a distal end region of the body portion 152. Collectively, the outer coupling member 162 and the inner tubular member 164 may form a male luer connection 148. The inner tubular member 164 may be coaxially disposed within the outer coupling member 162. Further, the outer coupling member 162 may be rotatably coupled to the body portion 152 or the inner tubular member 164. This may allow the inner tubular member 164 to be disposed within the lumen of another connection member (for example, but not limited to, the second connection member 140) while the outer coupling member 162 rotates to releasably secure the first connection member 138 to the additional connection member. In other examples, the entire first connection member 138 may rotate to releasably secure the first connection member 138 to the additional connection member. An outer diameter of the inner tubular member 164 may decrease from a proximal end region to the distal end 156 thereof. In some cases, the outer diameter may be sloped or tapered to form a friction fit with the inner surface of the second connection member 140 to form a fluid tight (e.g., air-light and liquid-tight) seal therewith. The outer diameter of the inner tubular member 164 may vary in other manners or may be substantially uniform as desired.

The outer coupling member 162 may include one or more internal helical recesses 166 configured to threadably engage one or more threads 178 of the second connection member 140. The outer coupling member 162 may be configured to be disposed over the outer surface of the proximal end region of the second connection member 140 while the inner tubular member 164 may be configured to be received within the lumen 176 of the second connection member 140 when the first connection member 138 is coupled with the second connection member 140. While the first and second connection members 138, 140 are shown and described as having a threaded engagement which can allow the first connection member 138 to be selectively and removably attached to the second connection member 140, other connection mechanisms may be used as desired. Some illustrative connection mechanisms may include, but are not limited to, friction fits, snap fits, a bayonet style connection, etc.

In some embodiments, the first connection member 138 may include a gripping region 168 disposed between the proximal end 154 and the outer coupling member 162. The gripping region 168 may extend radially beyond a radial extent of the proximal end 154, although this is not required. The gripping region 168 may be free from rotation relative to the proximal end 154 of the first connection member 138. In some cases, the gripping region 168 may include texturing, grooves, protrusions, etc. configured to increase the gripability of the device.

The second connection member 140 may include a body portion 170 extending from a first or proximal end 172 to a second or distal end 174. The body portion 170 may be formed as a single monolithic structure or may be formed from two or more components coupled to one another. A lumen 176 may extend through the first connection member 138 from the proximal end 172 to the distal end 174 thereof. The lumen 176 may have a diameter that varies along the length thereof. In some cases, the lumen 176 may include a first diameter 175 adjacent to the proximal end 172 of the second connection member 140 and a second diameter 177 adjacent to the distal end 174 of the second connection member 140. In some cases, the first diameter 175 may be greater than the second diameter 177. The portion of the lumen 176 having the first diameter 175 may extend distally from the proximal end 172 of the body portion 170. In some cases, the first diameter 175 may be approximately 0.189 inches (4.801 mm) with a length of approximately 0.5 inches (12.7 mm). It is contemplated that the first diameter 175 of the lumen 176 is not limited to a particular size but may be sized to receive a portion of the collet 142. Further, the portion of the lumen 176 having the first diameter 175 may have a length less than or greater than 0.5 inches (12.7 mm), as desired. The portion of the lumen 176 having the second diameter 177 may extend proximally from the distal end 174 of the body portion 170. In some cases, the diameter of the lumen 176 may transition from the first diameter 175 to the second diameter 177 in an abrupt or stair-step manner. In other examples, the diameter of the lumen 176 may transition from the first diameter 175 to the second diameter 177 in a gradual or tapered manner. In yet other examples, the diameter of the lumen 176 may be substantially constant along a length of the lumen 176. For example, the lumen 176 may have the first diameter 175 along an entire length thereof. It is contemplated that in some cases the first diameter 175 of the lumen 176 may be larger than a lumen of a standard luer fitting to allow a portion of the collet 142 to be disposed within the lumen 176.

The second connection member 140 may include one or more threads 178 extending radially from an outer surface of the body portion 170. In some embodiments, the one or more threads 178 may be adjacent to the proximal end 172 of the body portion 170. In some examples, the one or more threads 178 may be interrupted or discontinuous. In other examples, the one or more threads 178 may be helically continuous about the body portion 170. While the second connection member 140 is illustrated as having a single thread 178, in some examples, the second connection member 140 may have less than one thread (e.g., zero, less than one, etc.) or more than one thread 178, as desired. In some cases, the proximal end region of the second connection member 140 may form a female luer connection 146.

The second connection member 140 may further include an outer coupling member 180 and an inner tubular member 182 adjacent to a distal end region of the body portion 170. Collectively, the outer coupling member 180 and the inner tubular member 182 may form a male luer connection 150. The inner tubular member 182 may be coaxially disposed within the outer coupling member 180. Further, the outer coupling member 180 may be rotatably coupled to the body portion 170 or the inner tubular member 182. This may allow the inner tubular member 182 to be disposed within the lumen of another connection member (for example, but not limited to, the handle 20 of the catheter) while the outer coupling member 180 rotates to releasably secure the second connection member 140 to the additional connection member. In other examples, the entire second connection member 140 may rotate to releasably secure the second connection member 140 to the additional connection member. An outer diameter of the inner tubular member 182 may decrease from a proximal end region to the distal end 174 thereof. In some cases, the outer diameter may be sloped or tapered to form a friction fit with the inner surface of the handle 20 to form a fluid tight (e.g., air-light and liquid-tight) seal therewith. The outer diameter of the inner tubular member 182 may vary in other manners or be substantially uniform as desired.

The outer coupling member 180 may include one or more internal helical recesses 184 configured to threadably engage one or more threads 42 of the guidewire port 38 of the handle 20 of the catheter 10. The outer coupling member 180 may be configured to be disposed over the outer surface of the proximal end region of the guidewire port 38 while the inner tubular member 182 may be configured to be received within a lumen of the guidewire port 38 when the second connection member 140 is coupled with the handle 20 of the catheter 10. While the second connection member 140 and the guidewire port 38 are shown and described as having a threaded engagement which can allow the second connection member 140 to be selectively and removably attached to the handle 20, other connection mechanisms may be used as desired. Some illustrative connection mechanisms may include, but are not limited to, friction fits, snap fits, a bayonet style connection, etc. It is further contemplated that the second connection member 140 may be coupled to other portions of the catheter 10 or handle 20, if so desired.

In some embodiments, the second connection member 140 may include a gripping region 186 disposed between the proximal end 172 and the outer coupling member 180. The gripping region 186 may extend radially beyond a radial extent of the proximal end 172, although this is not required. The gripping region 186 may be free from rotation relative to the proximal end 172 of the second connection member 140. In some cases, the gripping region 186 may include texturing, grooves, protrusions, etc. configured to increase the gripability of the device.

The collet 142 may extend from a first or proximal end 188 to a second or distal end 190. A lumen 192 may extend from the proximal end 188 to the distal end 190. The lumen 192 may be sized to allow the mandrel 120 to pass therethrough. The collet 142 may include a proximal end region 196 having a first outer diameter less than a second outer diameter of a distal end region 194 of the collet 142. It is contemplated that at least some of the distal end region 194 of the collet 142 may have an outer diameter that is similar in size to or less than the first diameter 175 of the lumen 176 of the second connection member 140. For example, the collet 142 may be sized and shaped to be inserted into a proximal end 172 of the second connection member 140 and moved proximally until the distal end region 194 of the collet 142 contacts the smaller diameter 177 portion of the lumen 176.

The collet 142 may include one or more longitudinally extending slits 198 extending through a sidewall of the collet 142. While not explicitly shown, the collet 142 may include four slits 198. The slits 198 may be uniformly spaced about a circumference of the collet 142. For example, the slits 198 may be circumferentially spaced approximately 90° from another. However, this is not required. In some cases, the slits 198 may be eccentrically spaced. It is further contemplated that when more than one slit 198 is provided, the collet 142 may be divided into a plurality of radially actuatable segments 199a, 199b, 199c, 199d configured to allow the collet 142 to radially expand or radially compress along the length of the two or more slits 198. The number of segments 199a-d may depend on the number of slits 198. For example, when four slits 198 are provided, the distal end region 194 of the collet 142 may be divided into four segments. Each segment 199a-d may be spaced from adjacent segments 199a-d by the slits 198. The inner surface of the segments 199a-199d may be curved to generally conform to an outer surface of the mandrel 120.

The one or more slits 198 may extend less than an entire length of the collet 142. For example, the one or more slits 198 may extend proximally from the distal end 190 of the collet 142 and may terminate distal to the proximal end 188 of the collet 142. The one or more slits 198 may extend from an outer surface of the collet 142 to the lumen 192 thereof to allow the collet 142 to radially expand or radially compress along the length of the one or more slits 198. It is contemplated that the collet 142 may include any number of slits 198 desired. For example, the collet 142 may include one, two, three, four, or more slits.

In some embodiments, an outer diameter of the distal end region 194 may gradually increase from the distal end 190 of the collet 142 to a constant diameter region 195. The outer diameter of the collet 142 may reduce in a tapered or abrupt stair-step manner from the constant diameter region 195 to the outer diameter of the proximal end region 196. It is contemplated that the tapered region of the distal end region 194 may facilitate insertion of the collet 142 into the lumen 176 of the second connection member 140. The outer diameter of the proximal end region 196 may be substantially constant from the proximal end 188 of the collet 142 to the increased diameter region 195 of the distal end region 194. The proximal end region 196 may have an outer diameter that is less than a diameter of the lumen 158 of the first connection member 138 such that the proximal end region 196 may extend into the lumen 158 of the first connection member 138 with the proximal coupling assembly 122 is assembled.

FIGS. 5-7 illustrate side views of the proximal end region of the catheter 10 and the proximal coupling assembly 122 as the proximal coupling assembly 122 is assembled and connected to the catheter 10. To assemble the proximal coupling assembly 122, the second connection member 140 may be secured to the catheter 10. For example, the inner tubular member 182 may be inserted into a lumen of the handle 20 and the outer coupling member 180 may be disposed over an outer surface of a port, such as, but not limited to, the guidewire port 38. The outer coupling member 180 may be rotated to engage the internal threads 184 of the second connection member 140 with the external threads 42 of the guidewire port 38. It is contemplated that a mandrel 120, such as, but not limited to, a stainless-steel mandrel, may be inserted through a lumen (in some cases, the guidewire lumen 22) of the catheter 10 prior to coupling the second connection member 140 to the catheter 10. In such an instance, the second connection member 140 may be advanced over the mandrel 120 such that the mandrel 120 extends through the lumen 176 of the second connection member 140. However, this is not required. In some cases, the mandrel 120 may be inserted through the second connection member 140 and the catheter 10 after the second connection member 140 is coupled to the catheter 10. As will be described in more detail herein, the mandrel 120 may extend distally beyond a distal end of the catheter 10.

The collet 142 may also be positioned over the mandrel 120 such that the mandrel 120 is disposed within the lumen 192 of the collet 142. The collet 142 may be distally advanced over the mandrel 120 and at least partially into the lumen 176 of the second connection member 140, as shown in FIG. 6. It is contemplated that the transition in diameter from the first larger diameter 175 to the second smaller diameter 177 may limit distal movement of the distal end region 194 of the collet 142 within the lumen 176 of the second connection member 140. Said differently, the transition in diameter of the lumen 176 from a larger diameter to a smaller diameter may provide a mechanical stop to limit distal advancement of the collet 142 within the lumen 176 of the second connection member 140. For example, the distal end region 194 of the collet 142 may have an outer diameter that is greater than the second diameter 177 of the lumen 176 of the second connection member 140.

Next, the first connection member 138 may be positioned over the mandrel 120 such that the mandrel 120 is at least partially disposed within the lumen 158 of the first connection member 138. The first connection member 138 may be distally advanced over the mandrel 120 until the inner tubular member 164 is disposed within the lumen 176 of the second connection member 140 and the outer coupling member 162 is disposed over an outer surface of the second connection member 140, as shown in FIG. 7. The outer coupling member 162 may be rotated to engage the internal threads 166 of the first connection member 138 with the external threads 178 of the second connection member 140. It is contemplated that the proximal end region 196 of the collet 142 may be disposed within the lumen 158 of the first connection member 138 while the distal end region 194 of the collet 142 is disposed within the lumen 176 of the second connection member 140. For example, the outer diameter of the proximal end region 196 of the collet 142 may be less than the diameter 159 of the lumen 158 such that the inner tubular member 164 may be disposed over the proximal end region 196 of the collet 142 while the distal end 156 of the first connection member 138 abuts or is adjacent to the distal end region 194 of the collet 142. As the outer coupling member 162 is rotated and secured to the female luer lock 146 of the second connection member 140, the female luer lock 146 exerts a radially inward force on the distal end region 194 of the collet 142. This radially inward force may bias or push the segments 199a-d radially inwards towards the mandrel 120. The segments 199a-d may grip the mandrel 120 to secure the mandrel 120 relative to the catheter 10 and preclude axial and/or rotational movement of the mandrel 120 relative to the catheter 10.

The assembled proximal coupling assembly 122, mandrel 120, and catheter 10 may then be coupled to the drive unit 112 of the system 100. FIG. 8 is a side view of the proximal coupling assembly 122 coupled to the drive unit 112. The drive unit 112 may include a housing 113 configured to be rotated by the motor 114. The housing 113 may include a pin or coupling member 115 extending distally from an end surface thereof. The female luer lock 144 of the first connection member 138 may be disposed over the coupling member 115 in a press fit to secure the proximal coupling assembly 122 to the drive unit 112. It is contemplated that other coupling mechanisms may be used to secure the proximal coupling assembly 122 to the drive unit 112, as desired. Some illustrative coupling mechanisms may include, but are not limited to, snap fits, friction fits, threaded engagements, bayonet style locking mechanisms, as the like. Once the female luer lock 144 is coupled to the coupling member 115, rotation of the housing 113 may be translated to rotation of the proximal coupling assembly 122 and the catheter 10.

The catheter 10 may also be rotationally secured to the fixture 102 adjacent the distal end thereof. FIG. 9 is a perspective view of the distal end region of the catheter 10 secured to the second end region 110 of the fixture 102. The distal support member 118 may include a support housing 200 configured to be releasably secured to the mounting rail 106. Positioned on top of the support housing 200 may be a first gear housing 202 and a second gear housing 252. The first gear housing 202 may be releasably coupled to the second gear housing 252 via one or more fixation members 204a, 204b. In some examples, the first gear housing 202 may be movably coupled to the second gear housing 252. The one or more fixation members 204a, 204b may be set screws, screws, bolts, pins, or the like. The first gear housing 202 may include a recess 206 having a rotatable wheel or gear 208 disposed therein. The rotatable wheel 208 may be movably coupled to a mounting assembly 210 and/or the first gear housing 202. The rotatable wheel 208 may be coupled to the mounting assembly 210 and/or the first gear housing 202 in manner which translates the rotational movement of the wheel 208 to linear movement of the mounting assembly 210 and/or the first gear housing 202. For example, the rotatable wheel 208 may be coupled to the mounting assembly 210 using a slide crank mechanism, a scotch yoke mechanism, a rack and pinion, or the like. As the rotatable wheel 208 is rotated, the mounting assembly 210 and/or the first gear housing 202 may be axially displaced along the Z axis generally perpendicular to the longitudinal (X) axis of the mounting rail 106 (e.g., up and/or down), as shown at arrow 254. Rotation of the rotatable wheel 208 in a first direction may move the mounting assembly 210 and/or the first gear housing 202 in a direction towards the mounting rail 106 while rotation of the rotatable wheel 208 in a second direction, opposite the first direction, may move the mounting assembly 210 and/or the first gear housing 202 axially in a direction away from the mounting rail 106.

The second gear housing 252 may be releasably coupled to the support housing 200 via one or more fixation members (not explicitly shown). The one or more fixation members may be set screws, screws, bolts, pins, or the like. A rotatable adjustment knob 256 may extend from the second gear housing 252. The adjustment knob 256 may be movably coupled to the first gear housing 202. The adjustment knob 256 may be coupled to the first gear housing 202 in manner which translates the rotational movement of the adjustment knob 256 to linear movement of the first gear housing 202 and thus the mounting assembly 210. For example, the adjustment knob 256 may be coupled to the first gear housing 202 using a cylindrical cam, a lead screw assembly, or the like. As the adjustment knob 256 is rotated, the first gear housing 202 (and the mounting assembly 210 coupled thereto) may be axially displaced along an axis generally parallel to the longitudinal axis of the mounting rail 106, as shown at arrow 212. Rotation of the adjustment knob 256 in a first direction may move the first gear housing 202 (and the distal coupling assembly 222) axially in a direction towards the second end of the mounting rail 106 while rotation of the adjustment knob 256 in a second direction, opposite the first direction, may move the first gear housing 202 (and the distal coupling assembly 222) axially in a direction towards the first end of the mounting rail 106.

The mounting assembly 210 may include a top portion 214 of the first gear housing 202 and a mounting block 216. The top portion 214 may form an upper plate of the first gear housing 202 and may be movably coupled to the rotatable wheel 208. The mounting block 216 may be secured to the top portion 214 such that mounting block 216 moves with the first gear housing 202 (e.g., axial motion of the first gear housing 202 along the X or Z axis is translated to axial motion of the mounting block 216 along the X or Z axis). The mounting block 216 may be releasably coupled to the top portion 214 via one or more fixation members 218a, 218b. The one or more fixation members 218a, 218b may be set screws, screws, bolts, pins, or the like.

One or more bearings 220a, 220b may be coupled to the second mounting block 216. While the fixture 102 is illustrated as including two bearings 220a, 220b, the fixture 102 may include only one bearing or more than two bearings, as desired. The bearings 220a, 220b may be releasably coupled to the second mounting block 216 via one or more fixation members (not explicitly shown). The one or more fixation members may be set screws, screws, bolts, pins, or the like. The bearings 220a, 220b may be configured to receive a second end region of a distal coupling assembly 222 therein. The bearings 220a, 220b may be configured to allow for free rotation of the distal coupling assembly 222 about the longitudinal axis 36 of the catheter 10. For example, the distal end region of the catheter 10 may be coupled to the distal coupling assembly 222 to freely rotate as the motor 114 actively rotates the proximal end region of the catheter 10. It is contemplated that each rotation of the proximal end region of the catheter 10 results in one rotation of the distal end region of the catheter 10 (e.g., a direct drive system).

FIG. 10 illustrates a partially exploded perspective view of the illustrative bearings 220a, 220b and distal coupling assembly 222. Each bearing 220a, 220b may include a first ring 226a, 226b and a second ring 230a, 230b concentric with the first ring 226a, 226b. Rolling elements (not explicitly shown) may be placed between the concentric rings 226a, 230a of the first bearing 220a and between the concentric rings 226b, 230b of the second bearing 220b. The rolling elements may be enclosed beneath a cover 228a, 228b. An aperture 224a, 224b may extend through the innermost concentric ring 226a, 226b.

The distal coupling assembly 222 may extend from a first end 232 to a second end 234. Generally, the distal coupling assembly 222 may include a chuck 236, a cap 238, a body 240, and an elongate member or stem 242. The chuck 236 may be a clamp configured to hold the mandrel 120 (see, for example, FIG. 9) between one or more jaws 244a, 244b. The chuck 236 may include any number of jaws 244a, 244b configured to exert a gripping force on the mandrel 120. For example, the chuck 236 may include two, three, four, five, or more jaws. In some cases, the jaws 244a, 244b may be separated by gaps 246. It is contemplated that the number of gaps 246 may be determined by the number of jaws 244a, 244b. For example, a chuck having three jaws may have three gaps. An inner surface of the jaws 244a, 244b may be curved to conform to an outer surface of the mandrel 120

The cap 238 may be movably coupled to and disposed over the chuck 236 as well as the body 240. For example, the cap 238 may be threadably engaged with a threaded region 248 of the body 240. Rotation of the cap 238 in a first direction may move the cap 238 towards the first end 232 of the distal coupling assembly 222 while movement of the cap 238 in a second direction, opposite the first direction, may move the cap 238 towards the second end 234 of the distal coupling assembly 222. As the cap 238 is moved towards the second end 234 of the distal coupling assembly 222, the cap 238 may cause the jaws 244a, 244b to radially compress. This may allow the jaws 244a, 244b to grip and hold the mandrel 120. As the cap 238 is moved towards the first end 232 of the distal coupling assembly 222, the cap 238 may release its biasing force on the jaws 244a, 244b and the jaws 244a, 244b may radially expand allowing the mandrel 120 to be removed from the chuck 236. The reverse configuration is also contemplated in which movement of the cap 238 towards the first end 232 secures the mandrel 120 within the chuck 236 and movement of the cap 238 towards the second end 234 releases the mandrel 120 from the chuck 236.

The body 240 may be an elongated shaft extending from a first end disposed within the cap 238 to a second end 250. The threaded region 248 may be adjacent to the first end of the body 240. The stem 242 may extend from the second end 250 of the body 240 to the second end 234 of the distal coupling assembly 222. The stem 242 may have an outer diameter sized to be received within the apertures 224a, 224b of the bearings 220a, 220b. In some cases, the outer diameter of the stem 242 may be approximately the same as the diameter of the apertures 224a, 224b so that the stem 242 forms a friction or press fit with the apertures 224a, 224b.

To assemble the catheter 10 with the distal support member 118, the mandrel 120 may be advanced distally through a lumen (in some cases, the guidewire lumen 22) of the catheter 10 until the mandrel 120 exits the distal end of the catheter 10. The second end of the mandrel 120 may then be inserted into the chuck 232 (e.g., within a central opening defined by the radially expanded jaws 244a, 224b). The cap 238 may be rotated and axially displaced towards the second end 23 of the distal coupling assembly 222 to radially compress the jaws 244a, 244b and cause the jaws 244a, 244b to grip the mandrel 120. The stem 242 of the distal coupling assembly 222 may be inserted into the apertures 224a, 224b of the bearings 220a, 220b prior to coupling the mandrel 120 with the distal coupling assembly 222. In other examples, the stem 242 of the distal coupling assembly 222 may be inserted into the apertures 224a, 224b of the bearings 220a, 220b after coupling the mandrel 120 with the distal coupling assembly 222. The mandrel 120 may form a friction fit with the lumen of the catheter 10 adjacent a distal end region thereof such that the distal end region of the catheter 10 moves with the mandrel 120. In some cases, the second end of the mandrel 120 may be coupled to the distal coupling assembly 222 and/or the bearings 220a, 220b prior to affixing the first end of the mandrel 120 with the coupling member 115. In other examples, the first end of the mandrel 120 may be coupled to collet 142 prior to coupling the second end of the mandrel 120 with the coupling member 115.

It is contemplated that the distal support member 118 may be positioned axially along the mounting rail 106 to allow the distal coupling assembly 222 to be easily assembled with the bearings 220a, 220b and the mandrel 120 to be coupled to the distal coupling assembly 222. However, some slack may remain in the elongate shaft 10 when both the first end of the mandrel 120 is coupled to the proximal coupling assembly 122 (and the drive unit 112) and the second end of the mandrel 120 is coupled to the distal coupling assembly 222 (and the bearings 220a, 220b). It is contemplated that the adjustable knob 256 may be actuated once the mandrel 120 is fixed relative to the catheter 10 and the proximal coupling assembly 122 and the distal coupling assembly 222 are fixed to the fixture 102. The adjustable knob 256 may be rotated to move the mounting assembly 210 and the distal coupling assembly 222 in a direction towards the second end region 110 of the mounting rail 106 to remove any slack, sagging, or curvature from the catheter 10 and/or the balloon 14. Said differently, the mandrel 120 may be affixed at each thereof and the second end axially displaced to pull it taut and remove sagging from the balloon 14. It is contemplated that either or both the distal support member 118 and the adjustable knob 256 may be utilized to pull the mandrel 120 taut. For example, the distal support member 118 may provide for gross adjustments while the actuatable knob 256 may allow for finer, more precise adjustments. However, this is just an example. In other examples, the distal support member 118 may be axially displaced along the mounting rail 106 to pull the mandrel 120 taut. In yet other examples, the adjustable knob 256 may be used to axially displace the mounting assembly 210 to pull the mandrel 120 taut. Further, the rotatable wheel 208 may be actuated to adjust a height of the distal coupling assembly 222 along the Z axis. Once the catheter 10 is affixed within the fixture 102, the coating may be dispensed via the coating system 104. When the coating process is complete, the catheter 10 may be removed from the fixture 102 by reversing the assembly process described herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fixture for holding a medical device, the fixture comprising:
a mounting rail extending from a first end region to a second end region;
a drive unit movably coupled to the mounting rail adjacent the first end region thereof;
a proximal coupling assembly removably coupled to the drive unit, the proximal coupling assembly comprising:
a first connection member;
a second connection member releasably coupled to the first connection member; and
a collet disposed within a lumen of the first connection member and within a lumen of the second connection member;
a support member movably coupled to the mounting rail adjacent the second end region thereof;
a distal coupling assembly removably coupled to the support member, the distal coupling assembly comprising:
a chuck;
a cap;
a body; and
an elongate member; and
a mandrel configured to extend through a lumen of a medical device, the mandrel having a first end configured to be releasably coupled to the proximal coupling assembly and a second end configured to be releasably coupled to the distal coupling assembly.

2. The fixture of claim 1, wherein the mandrel extends through the lumen of the second connection member and a lumen of the collet.

3. The fixture of claim 2, wherein when the first connection member is coupled with the second connection member, the second connection member exerts a radially compressive force on the collet.

4. The fixture of claim 2, wherein when the first connection member is coupled with the second connection member, the collet exerts a radially compressive force on an outer surface of the mandrel.

5. The fixture of claim 1, wherein the collet has a first outer diameter adjacent to a first end thereof and a second outer diameter adjacent a second end thereof, the second outer diameter greater than the first outer diameter.

6. The fixture of claim 1, wherein a distal end region of the collet comprises a plurality of radially actuated segments.

7. The fixture of claim 1, wherein the second end of the mandrel is configured to be received within the chuck of the distal coupling assembly.

8. The fixture of claim 1, wherein the support member comprises a mounting assembly movably coupled to an actuatable knob.

9. The fixture of claim 8, wherein actuation of the actuatable knob is translated to linear moment of the mounting assembly.

10. The fixture of claim 8, wherein actuation of the actuatable knob in a first direction is configured to move the distal coupling assembly towards the second end region of the mounting rail and actuation of the actuatable knob in a second direction opposite the first direction is configured to move the distal coupling assembly towards the first end region of the mounting rail.

11. The fixture of claim 1, wherein the support member is axially displaced along the mounting rail to pull the mandrel taut.

12. The fixture of claim 8, wherein the mounting assembly is axially displaced along an axis parallel to the mounting rail to pull the mandrel taut.

13. The fixture of claim 1, wherein the drive unit is configured to actively rotate the proximal coupling assembly.

14. A fixture for holding a medical device, the fixture comprising:
a mounting rail extending from a first end region to a second end region;
a drive unit movably coupled to the mounting rail adjacent the first end region thereof;
a proximal coupling assembly removably coupled to the drive unit, the proximal coupling assembly comprising:
a first connection member;
a second connection member releasably coupled to the first connection member; and
a collet disposed within a lumen of the first connection member and within a lumen of the second connection member;
a support member movably coupled to the mounting rail adjacent the second end region thereof;
a distal coupling assembly removably coupled to the support member; and
a mandrel configured to extend through a lumen of a medical device, the mandrel having a first end configured to be releasably coupled to the proximal coupling assembly and a second end configured to be releasably coupled to the distal coupling assembly;
wherein actuation of the distal coupling assembly and/or the support member is configured to pull the mandrel taut.

15. The fixture of claim 14, wherein when the first connection member is coupled with the second connection member, the collet exerts a radially compressive force on an outer surface of the mandrel.

16. A fixture for holding a medical device, the fixture comprising:
a mounting rail extending from a first end region to a second end region;
a drive unit movably coupled to the mounting rail adjacent the first end region thereof;
a proximal coupling assembly removably coupled to the drive unit, the proximal coupling assembly comprising:
a first connection member;
a second connection member releasably coupled to the first connection member; and
a collet disposed within a lumen of the first connection member and within a lumen of the second connection member;
a support member movably coupled to the mounting rail adjacent the second end region thereof;
a distal coupling assembly removably coupled to the support member, the distal coupling assembly comprising:
a chuck;
a cap;
a body; and
an elongate member; and
a mandrel configured to extend through a lumen of a medical device, the mandrel having a first end configured to be releasably coupled to the proximal coupling assembly and a second end configured to be releasably coupled to the distal coupling assembly;
wherein actuation of the distal coupling assembly and/or the support member is configured to pull the mandrel taut.

17. The fixture of claim 16, wherein the first connection member comprises a female luer lock adjacent a first end thereof and a male luer lock adjacent a second end thereof.

18. The fixture of claim 16, wherein the lumen of the first connection member has a varying diameter along a length thereof.

19. The fixture of claim 16, wherein the second connection member comprises a female luer lock adjacent a first end thereof and a male luer lock adjacent a second end thereof.

20. The fixture of claim 16, wherein the lumen of the second connection member has a first diameter adjacent a proximal end thereof and a second diameter adjacent to a distal end thereof, the first diameter greater than the second diameter.
